(19) United States
(12) Patent Application Publication
(10) Pub. No.: US 2018/0371871 A1
(43) Pub. Date: Dec. 27, 2018

(54) Methods Of Drawing Out Oils And Fats From Solid Material Using Chlorine Dioxide

(71) Applicant: Sabre Intellectual Property Holdings LLC, Slingerlands, NY (US)

(72) Inventors: John Y. Mason, Slingerlands, NY (US); Madeline C. Bette, Loudonville, NY (US); Kevin M. Bette, Loudonville, NY (US)

(21) Appl. No.: 16/063,650

(22) PCT Filed: Dec. 16, 2016
(86) PCT No.: PCT/US2016/067236

§ 371 Date: Jun. 18, 2018

Related U.S. Application Data
(60) Provisional application No. 62/269,812, filed on Dec. 18, 2015.

(51) Int. Cl. E21B 37/06; B08B 9/08; B09B 3/00; C09K 8/594

(52) U.S. Cl. CPC E21B 37/06; C09K 8/594; B09B 3/0058; B08B 9/08

(57) ABSTRACT

Provided herein are methods of drawing out oils and/or fats (e.g., hydrocarbons) from a solid material (such as, e.g., metal or rock, e.g., a hydrocarbon bearing geologic formation). The methods comprise fumigating the solid material with a gas containing chlorine dioxide, thereby drawing out oil and/or fat from the solid material. The methods can be used, e.g., to clean solid materials used in industry and to enhance the recovery of crude oil and/or natural gas from petroleum wells.

### RELATED APPLICATIONS

This application claims priority to U.S. Patent Application No. 62/269,812 filed on Dec. 18, 2015, the entire contents of which are hereby incorporated herein by reference.

### BACKGROUND

Many solid materials, such as, for example, metals, concrete, brick, wood, plaster, and ceramics, can soak up oils and fats. Removing oils and fats from such materials can require complex and expensive cleaning processes. The present application provides new methods for effectively drawing out oils and fats from solid materials. Methods disclosed herein can also be used to enhance recovery of hydrocarbon from hydrocarbon bearing formations.

### SUMMARY

In one aspect provided herein is a method of drawing out oil and/or fat from a solid material, the method comprising fumigating the solid material with a gas containing chlorine dioxide, thereby drawing out oil and/or fat from the solid material.

In some embodiments, the fumigating is conducted at a concentration×time (CT) value of at least 3000 ppmᵥ-hours.

In some embodiments, the fumigating is conducted at a concentration×time (CT) value of at least 9000 ppmᵥ-hours.

In some embodiments, the fumigating is conducted at a concentration×time (CT) value of 3,000 to 500,000 ppm hours.

In some embodiments, the solid material has previously been exposed to the oil and/or the fat and has absorbed the oil and/or the fat.

In some embodiments, the method can draw out at least 5, 10, 15, 20, 25, 30, 40, 50, 60, 70, 75, 80, 90, or 95% by weight of the absorbed oil and/or fat. In some embodiments, the method can draw out at least 5, 10, 15, 20, 25, 30, 40, 50, 60, 70, 75, 80, 90, or 95% by volume of the absorbed oil and/or fat.

In some embodiments, the solid material is metal, rock, sand, clay, concrete, brick, wood, plaster, drywall or a ceramic.

In some embodiments, the metal is iron or an iron alloy.

In some embodiments, the iron alloy is cast iron or steel.

In some embodiments, the oil is a hydrocarbon.

In some embodiments, the oil and/or fat is plant-derived or animal derived.

Also provided herein is a method of cleaning a solid material, the method comprising fumigating the solid material with a gas containing chlorine dioxide, thereby drawing out oil and/or fat from the solid material.

In some embodiments, the solid material is a petroleum tanker, e.g., a crude tanker (e.g., an ultra large crude carrier) or a product tanker. In some embodiments, the solid material is a line or other equipment that is used for processing or transport of petroleum products.

In some embodiments, the method further comprises removing the drawn out oil and/or fat from the surface of the solid material.

In some embodiments, the removing is performed after the fumigating. In some embodiments, the removing is performed within 12, 6, 5, 4, 3, or 2 hours after the fumigating. In some embodiments, the removing is performed within 1 hour after the fumigating.

In some embodiments, the removing is performed during or immediately after the fumigating.

A method of drawing out hydrocarbon from a hydrocarbon bearing formation, the method comprising fumigating the hydrocarbon bearing formation with a gas containing chlorine dioxide, thereby drawing out hydrocarbon from the hydrocarbon bearing formation. The hydrocarbon can comprise, e.g., crude oil, natural gas, bitumen, or tar.

In some embodiments, the fumigating comprises introducing the gas containing chlorine dioxide into the wellbore of a well that penetrates the hydrocarbon bearing formation.

In some embodiments, the gas containing chlorine dioxide comprises air.

In some embodiments, the gas containing chlorine dioxide further comprises carbon dioxide gas, nitrogen gas, natural gas, or a combination thereof.

In some embodiments, the gas containing chlorine dioxide further comprises hydrogen chloride gas.

In some embodiments, the method further comprises introducing a corrosion inhibitor into the wellbore. In some embodiments, the introducing is performed before the fumigating.

In some embodiments, the method further comprises removing the drawn out hydrocarbon from the hydrocarbon bearing formation. In some embodiments, the removing is performed within a time period disclosed herein. In some embodiments, the removing comprises contacting the hydrocarbon bearing formation with a washing fluid (e.g., a flushing medium).

In some embodiments, the method further comprises introducing a flushing medium into the hydrocarbon bearing formation (e.g., into a wellbore that penetrates the hydrocarbon bearing formation) and recovering at least a portion of the flushing medium.

In some embodiments, the flushing medium is introduced after the fumigating. In some embodiments, the flushing medium is introduced within 12, 6, 5, 4, 3, or 2 hours after the fumigating. In some embodiments, the flushing medium is introduced within 4 hours after the fumigating. In some embodiments, the flushing medium is introduced within 1 hour after the fumigating.

In some embodiments, the flushing medium is introduced immediately after the fumigating.

In some embodiments, the method enhances recovery of crude oil and/or natural gas from the well.

Also provided herein is a method of drawing out crude oil and/or natural gas from a hydrocarbon bearing formation. The method comprises fumigating the hydrocarbon bearing formation with a gas containing chlorine dioxide, thereby drawing out crude oil and/or natural gas from the hydrocarbon bearing formation. In some embodiments, the fumigating comprises introducing the gas containing chlorine dioxide into the wellbore of a well that penetrates the hydrocarbon bearing formation.

In some embodiments, the gas containing chlorine dioxide further comprises carbon dioxide gas, nitrogen gas, natural gas, or a combination thereof.

In some embodiments, the gas containing chlorine dioxide further comprises hydrogen chloride gas.

In some embodiments, the method further comprises introducing a corrosion inhibitor into the wellbore. In some embodiments, the introducing is performed before the fumigating.

In some embodiments, the method further comprises contacting the hydrocarbon bearing formation with a washing fluid (e.g., a flushing medium). In some embodiments, the contacting comprises introducing a flushing medium into the hydrocarbon bearing formation (e.g., into the wellbore) and recovering at least a portion of the flushing medium.

In some embodiments, the flushing medium is introduced after the fumigating. In some embodiments, the flushing medium is introduced within 12, 6, 5, 4, 3, or 2 hours after the fumigating. In some embodiments, the flushing medium is introduced within 4 hours after the fumigating. In some embodiments, the flushing medium is introduced within 1 hour after the fumigating.

In some embodiments, the flushing medium is introduced immediately after the fumigating.

### DETAILED DESCRIPTION

### Definitions

As used herein, singular terms such as “a,” “an,” or “the” include the plural, unless the context clearly indicates otherwise.

As used herein, a concentration-time value, also referred to as a “CT” or “CT value”, is the time-weighted average of chlorine dioxide concentration in parts per million by volume (ppmᵥ) multiplied by the exposure time in hours. In a plot of chlorine dioxide concentration versus exposure time in hours, the CT would equal the area under the curve. For example, if the time weighted average chlorine dioxide concentration over a 12-hour exposure period were 750 ppmᵥ, the CT would be 9,000 ppmᵥ-hr. Similarly, if the time weighted average chlorine dioxide concentration over a 3-hour exposure period were 3,000 ppmᵥ, the CT would still be 9,000 ppmᵥ-hr.

As used herein, a “biological contaminant” refers to a contaminant such as a virus, alga, protozoan, bacterium, or fungus. In some embodiments, the biological contaminant is an organism that can cause infectious disease in an animal (e.g., a human and/or a non-human animal).

As used herein, “carbon dioxide” refers to CO₂. The carbon dioxide can be gaseous carbon dioxide, supercritical carbon dioxide, or liquid carbon dioxide. In some embodiments, the carbon dioxide is carbon dioxide gas. In some embodiments, the carbon dioxide is supercritical carbon dioxide. In some embodiments, the carbon dioxide is liquid carbon dioxide.

As used herein, “damage” refers to an undesired residue that can arise from buildup of particles, fluids, and/or contaminants (e.g., bacteria or biomass) in a wellbore and in the immediate vicinity of the wellbore. Damage can be caused by foreign fluids or other matter introduced during petroleum industry operations. Substances that can be present in the damage include, for example, sulfides (e.g., iron sulfide), sulfur, polymers (e.g., polyacrylamides, carboxymethylcellulose, hydroxyethylcellulose, hydroxypropyl guar), xanthan gum, carbonates (e.g., calcium carbonate), hydrocarbons, paraffins, asphaltenes, bacteria, biofilm and/or biomass.

As used herein, “fumigating” a solid material with a gas containing chlorine dioxide means exposing the solid material to a gas that contains chlorine dioxide. Typically, the fumigating is performed in an enclosed volume.

As used herein, a “hydrocarbon” refers to any organic compound made up of only hydrogen and carbon (or a mixture of such organic compounds) as well as petroleum hydrocarbons such as crude oil, natural gas, bitumen and tar. Accordingly, the hydrocarbon can be one or more hydrocarbon compounds made up of only hydrogen and carbon, e.g., an aliphatic hydrocarbon (e.g., an aliphatic saturated hydrocarbon (e.g., a straight or branched chain aliphatic hydrocarbon, or a cycloalkane), an aliphatic unsaturated hydrocarbon (e.g., an alkene (olefin) or an alkyne (acetylene)), an aromatic hydrocarbon (e.g., an aromatic hydrocarbon having a single aromatic ring or two or more aromatic rings), or a mixture of such hydrocarbon compounds.

Hydrocarbon can include liquid, solid, semisolid, and/or gas components. In some embodiments, the hydrocarbon is in the form of a liquid or a gas at 20° C. and 760 mmHg (i.e., 1 atm). In some embodiments, the hydrocarbon is in the form of a liquid or a gas under the conditions present (e.g., when a method disclosed herein is performed). In some embodiments, the hydrocarbon is in the form of a liquid at 20° C. and 760 mmHg. In some embodiments, the hydrocarbon is in the form of a liquid (e.g., under the conditions present when a method disclosed herein is performed). In some embodiments, the hydrocarbon is a liquid or gas at 20° C. or has a melting point of 80° C. or less (at a pressure of 760 mm Hg). In some embodiments, the hydrocarbon is a liquid or gas at 20° C. or has a melting point of 50° C. or less (at a pressure of 760 mm Hg).

As used herein, a “hydrocarbon bearing formation” or “hydrocarbon bearing geologic formation” is a formation that can release hydrocarbons, e.g. crude oil and/or natural gas. Such a formation can include, e.g., source rock that generates or is capable of generating hydrocarbons and/or reservoir rock that accumulates hydrocarbons.

As used herein, “iron” can be iron in any oxidation state, such as, e.g., iron (II) or iron (III). Furthermore, iron can be any iron isotope, e.g., ⁵⁴Fe, ⁵⁶Fe, ⁵⁷Fe, ⁵⁸Fe, or a mixture thereof. Naturally occurring iron is generally a mixture of ⁵⁴Fe, ⁵⁶Fe, ⁵⁷Fe, and ⁵⁸Fe.

As used herein and in the art, “ppm” refers to parts per million. In the describing liquid solutions comprising chlorine dioxide, the present specification employs the term “ppm” to refer to parts per million by weight. As used herein, the term “ppmᵥ or ppmv” refers to parts per million by volume.

As used herein, the “percent,” “percentage” or “%” concentration of a component is intended to refer to the w/w % concentration unless the context indicates otherwise.

As used herein, the “solubility” of one substance in another is typically assessed under ambient conditions (preferably at a temperature of about 20° C. and at atmospheric pressure).

As used herein, “trimethylbenzene” can be, e.g., 1,2,3-trimethylbenzene, 1,2,4-trimethylbenzene, 1,3,5-trimethylebenzene, or any mixture of two or more of the foregoing forms.

As used herein, a “well” is a petroleum well. The well can be a production well that is used to extract oil and/or gas, and/or the well can be an injection well.

As used herein “xylene” can be, e.g., o-xylene, m-xylene, p-xylene, or any mixture of two or more of the foregoing forms of xylene. As used herein, “xylene” can also include commercially available forms of xylene that can contain up to 20% ethylbenzene in addition to m-xylene, o-xylene, and/or p-xylene. In some embodiments, the xylene is a commercially available xylene that contains 40-65% m-xylene and up to 20% each of o-xylene, p-xylene, and ethylbenzene.

### Introduction

In one aspect provided herein is a method of drawing out oil and/or fat from a solid material. The method comprises fumigating the solid material with a gas containing chlorine dioxide, thereby drawing out oil and/or fat from the solid material.

In another aspect provided herein is a method of cleaning a solid material. The method comprises fumigating the solid material with a gas containing chlorine dioxide, thereby drawing out oil and/or fat from the solid material.

In a further aspect provided herein is a method of drawing out hydrocarbon from a hydrocarbon bearing formation. The method comprises fumigating the hydrocarbon bearing formation with a gas containing chlorine dioxide, thereby drawing out hydrocarbon from the hydrocarbon bearing formation.

In a further aspect provided herein is a method of drawing out crude oil and/or natural gas from a hydrocarbon bearing formation. The method comprises fumigating the hydrocarbon bearing formation with a gas containing chlorine dioxide, thereby drawing out crude oil and/or natural gas from the hydrocarbon bearing formation.

Without wishing to be bound by theory, Applicant believes that the methods disclosed herein are effective because chlorine dioxide gas can penetrate or infuse into solid material, where it can contact and draw out oils and/or fats contained within the solid material.

Further information and embodiments relating to these aspects are provided throughout the present disclosure.

### Fumigating

Methods disclosed herein comprise fumigating with a gas containing chlorine dioxide. Fumigation methods are known in the art and are disclosed, e.g., in U.S. Pat. Nos. 7,678,388; 8,192,684; and 8,741,223; and in Canadian Patent No. 2,583,459. Chlorine dioxide gas can be produced using any means known in the art. A chlorine dioxide generator, such as, e.g., the chlorine dioxide generator described in U.S. Pat. No. 6,468,479 can be used to make chlorine dioxide gas. See, e.g., U.S. Pat. Nos. 6,645,457; 7,807,101; 8,192,684; and 8,741,223. Other methods and devices for generating chlorine dioxide are disclosed in, for example, U.S. Pat. Nos. 7,678,388; 5,290,524, and 5,234,678.

In some embodiments, the gas utilized in the fumigating further comprises air. The air can have humidity levels disclosed herein. In some embodiments, the gas consists of, or consists essentially of, chlorine dioxide and air. In some embodiments, the gas comprises nitrogen, oxygen, argon, and/or carbon dioxide.

In some embodiments, the gas comprises carbon dioxide. In some embodiments, the gas comprises, consists of, or consists essentially of, chlorine dioxide, air, and carbon dioxide.

In some embodiments, the gas consists of, or consists essentially of, chlorine dioxide gas and carbon dioxide.

In some embodiments, the fumigating is carried out in an enclosed volume. Preferably, the enclosed volume is sealed to prevent or minimize entry of air. In some embodiments, the enclosed volume is a wellbore. Sealing materials known in the art can be used to prevent or minimize entry of air into the enclosed volume.

In some embodiments, a slight negative pressure is maintained in the enclosed volume. In some embodiments, the negative pressure is a negative pressure of at least −0.005 inches of water (or more negative) (i.e., −0.009 mm Hg or more negative).

In embodiments, the gas is introduced into the enclosed volume to maintain a minimum chlorine dioxide concentration during the fumigating.

In some embodiments, the minimum chlorine dioxide concentration of the gas during the fumigating is at least 200 ppmᵥ; 500 ppmᵥ; 750 ppmᵥ; 1000 ppmᵥ; 1500 ppmᵥ; 2000 ppmᵥ; 3000 ppmᵥ; 4000 ppmᵥ; 5000 ppmᵥ; 6000 ppmᵥ; 7000 ppmᵥ; 8000 ppmᵥ; 9000 ppmᵥ; 10,000 ppmᵥ; 15,000 ppmᵥ; or 20,000 ppmᵥ.

In some embodiments, the minimum chlorine dioxide concentration of the gas during the fumigating is in the range of 200 ppmᵥ to 20,000 ppmᵥ.

In some embodiments, the minimum chlorine dioxide concentration of the gas during the fumigating is in the range of 500 ppmᵥ to 3,000 ppmᵥ.

In some embodiments, the minimum chlorine dioxide concentration of the gas during the fumigating is in the range of 500 ppmᵥ to 15,000 ppmᵥ; 1000 ppmᵥ to 15,000 ppmᵥ; 2000 ppmᵥ to 15,000 ppmᵥ; 3000 ppmᵥ to 15,000 ppmᵥ; 4000 ppmᵥ to 15,000 ppmᵥ; or 5000 ppmᵥ to 15,000 ppmᵥ.

In some embodiments, the minimum chlorine dioxide concentration of the gas during the fumigating is in the range of 500 ppmᵥ to 20,000 ppmᵥ; 1000 ppmᵥ to 20,000 ppmᵥ; 2000 ppmᵥ to 20,000 ppmᵥ; 3000 ppmᵥ to 20,000 ppmᵥ; 4000 ppmᵥ to 20,000 ppmᵥ; or 5000 ppmᵥ to 20,000 ppmᵥ.

At atmospheric pressure, chlorine dioxide gas becomes explosive at a concentration of about 110,000 ppmᵥ. In embodiments, the concentration of chlorine dioxide in the gas does not exceed 100,000 ppmᵥ. In embodiments, the concentration of chlorine dioxide in the gas does not exceed about 20,000 ppmᵥ, 25,000 ppmᵥ, 30,000 ppmᵥ, 40,000 ppmᵥ, 50,000 ppmᵥ, 60,000 ppmᵥ, 70,000 ppmᵥ, 80,000 ppmᵥ, or 90,000 ppmᵥ.

In embodiments, the maximum concentration of chlorine dioxide in the gas is in the range of 750 ppmᵥ to 20,000 ppmᵥ; 1000 ppmᵥ to 20,000 ppmᵥ; 2000 ppmᵥ to 20,000 ppmᵥ; 3000 ppmᵥ to 20,000 ppmᵥ; 4000 ppmᵥ to 20,000 ppmᵥ; 5000 ppmᵥ to 20,000 ppmᵥ; 6000 ppmᵥ to 20,000 ppmᵥ; 7000 ppmᵥ to 20,000 ppmᵥ; 8000 ppmᵥ to 20,000 ppmᵥ; 9000 ppmᵥ to 20,000 ppmᵥ; 10,000 ppmᵥ to 20,000 ppmᵥ; 15,000 ppmᵥ to 20,000 ppmᵥ; or 16,000 ppmᵥ to 20,000 ppmᵥ.

In some embodiments, the maximum concentration of chlorine dioxide in the gas is in the range of 750 ppmᵥ to 50,000 ppmᵥ; 1000 ppmᵥ to 50,000 ppmᵥ; 2000 ppmᵥ to 50,000 ppmᵥ; 3000 ppmᵥ to 50,000 ppmᵥ; 5000 ppmᵥ to 50,000 ppmᵥ; 10,000 ppmᵥ to 50,000 ppmᵥ; 15,000 ppmᵥ to 50,000 ppmᵥ; or 20,000 ppmᵥ to 50,000 ppmᵥ.

In some embodiments, the maximum concentration of chlorine dioxide in the gas is 750 ppmᵥ to 100,000 ppmᵥ; 1000 ppmᵥ to 100,000 ppmᵥ; 2000 ppmᵥ to 100,000 ppmᵥ; 3000 ppmᵥ to 100,000 ppmᵥ; 5000 ppmᵥ to 100,000 ppmᵥ; 10,000 ppmᵥ to 100,000 ppmᵥ; 15,000 ppmᵥ to 100,000 ppmᵥ; or 20,000 ppmᵥ to 100,000 ppmᵥ.

In some embodiments, the time weighted average concentration of chlorine dioxide in the gas is at least 750 ppmᵥ, 1000 ppmᵥ, 2000 ppmᵥ, 3000 ppmᵥ, 5000 ppmᵥ, 10,000 ppmᵥ, or 15,000 ppmᵥ.

In some embodiments, the time weighted average concentration of chlorine dioxide in the gas is up to 16,000 ppmᵥ, 17,000 ppmᵥ, 18,000 ppmᵥ, or 19,000 ppmᵥ. In some embodiments, the time weighted average concentration of chlorine dioxide in the gas is up to 20,000 ppmᵥ, 25,000 ppmᵥ, 30,000 ppmᵥ, 40,000 ppmᵥ, 50,000 ppmᵥ, 60,000 ppmᵥ, 70,000 ppmᵥ, or 80,000 ppmᵥ.

In some embodiments, the time weighted average concentration of chlorine dioxide in the gas is in the range of 750 ppmᵥ to 80,000 ppmᵥ; 750 ppmᵥ to 70,000 ppmᵥ; 750 ppmᵥ to 60,000 ppmᵥ; 750 ppmᵥ to 50,000 ppmᵥ; 750 ppmᵥ to 40,000 ppmᵥ; 750 ppmᵥ to 30,000 ppmᵥ; 750 ppmᵥ to 25,000 ppmᵥ; 750 ppmᵥ to 20,000 ppmᵥ; or 750 ppmᵥ to 15,000 ppmᵥ.

In some embodiments, the time weighted average concentration of chlorine dioxide in the gas is in the range of 5000 ppmᵥ to 80,000 ppmᵥ. In some embodiments, the time weighted average concentration of chlorine dioxide in the gas is in the range of 5000 ppmᵥ to 70,000 ppmᵥ. In some embodiments, the time weighted average concentration of chlorine dioxide in the gas is in the range of 5000 ppmᵥ to 60,000 ppmᵥ. In some embodiments, the time weighted average concentration of chlorine dioxide in the gas is in the range of 5000 ppmᵥ to 50,000 ppmᵥ. In some embodiments, the time weighted average concentration of chlorine dioxide in the gas is in the range of 5000 ppmᵥ to 40,000 ppmᵥ. In some embodiments, the time weighted average concentration of chlorine dioxide in the gas is in the range of 1000 ppmᵥ to 30,000 ppmᵥ. In some embodiments, the time weighted average concentration of chlorine dioxide in the gas is in the range of 1000 ppmᵥ to 25,000 ppmᵥ. In some embodiments, the time weighted average concentration of chlorine dioxide in the gas is in the range of 1000 ppmᵥ to 20,000 ppmᵥ.

In embodiments of the methods described herein, the methods comprise fumigating with a gas containing chlorine dioxide at a CT value that is effective to draw out oil and/or fat from the solid material. As a person of skill in the art will recognize, the chlorine dioxide can act not only to draw out oil and/or fat from the solid material; in some cases, the chlorine dioxide can also oxidize the solid material and/or contaminants (e.g., biological contaminants) that are present on the solid material. Accordingly, the CT that is required to draw out the oil and/or fat will increase if the solid material has demand for chlorine dioxide, e.g., if the solid material itself can be oxidized by chlorine dioxide (as an example, chlorine dioxide can oxidize iron (II) to iron (III)) or if the solid material has contaminants (e.g., biological contaminants such as a biofilm) that can be oxidized by the chlorine dioxide.

In embodiments, the methods comprise fumigating with a gas containing chlorine dioxide at a CT value of at least 3,000 ppmᵥ-hours, 4,000 ppmᵥ-hours, 5,000 ppmᵥ-hours, 6,000 ppmᵥ-hours, 7,000 ppmᵥ-hours, 8,000 ppmᵥ-hours, 9,000 ppmᵥ-hours, 10,000 ppmᵥ-hours, 20,000 ppmᵥ-hours, 30,000 ppmᵥ-hours, 40,000 ppmᵥ-hours, 50,000 ppmᵥ-hours, 60,000 ppmᵥ-hours, 70,000 ppmᵥ-hours, 75,000 ppmᵥ-hours, 80,000 ppmᵥ-hours, 90,000 ppmᵥ-hours, 100,000 ppmᵥ-hours, 110,000 ppmᵥ-hours, 120,000 ppmᵥ-hours, 130,000 ppmᵥ-hours, 140,000 ppm hours, or 150,000 ppm hours.

In embodiments, the CT value is up to 200,000 ppmᵥ-hours, 250,000 ppmᵥ-hours, 300,000 ppmᵥ-hours, 400,000 ppmᵥ-hours, or 500,000 ppmᵥ-hours. In embodiments, the CT value is up to 1,000,000 ppmᵥ-hours. In embodiments, the CT value is up to 2,000,000 ppmᵥ-hours. In embodiments, the CT value is up to 3,000,000 ppm hours.

In embodiments, the CT value is 3,000 to 500,000 ppmᵥ-hours. In embodiments, the CT value is 9000 to 500,000 ppmᵥ-hours.

In embodiments, the CT value is 5,000 to 500,000 ppmᵥ-hours. In embodiments, the CT value is 9000 to 500,000 ppmᵥ-hours.

In embodiments, the CT value is 20,000 to 500,000 ppmᵥ-hours. In embodiments, the CT value is 20,000 to 500,000 ppmᵥ-hours or 20,000 to 300,000 ppmᵥ-hours.

In embodiments, the CT value is 20,000 to 1,000,000 ppmᵥ-hours. In embodiments, the CT value is 20,000 to 2,000,000 ppm hours. In embodiments, the CT value is 20,000 to 3,000,000 ppmᵥ-hours.

In embodiments, the solid material (e.g., the hydrocarbon bearing formation) is exposed to the gas comprising chlorine dioxide for an exposure time of at least about 30 minutes. In embodiments, the material is exposed to the gas comprising chlorine dioxide for an exposure time of at least about 1 hour, 2 hours, 3 hours, 4, hours, 6 hours, 8 hours, or 12 hours. In embodiments, the material is exposed to the gas comprising chlorine dioxide for an exposure time of at least about 1 day. In embodiments, the material is exposed to the gas comprising chlorine dioxide for an exposure time of up to about 1 week.

In embodiments, the solid material (e.g., the hydrocarbon bearing formation) is exposed to the gas comprising chlorine dioxide for an exposure time of 30 minutes to 1 week. In embodiments, the material is exposed to the gas comprising chlorine dioxide for an exposure time of about 1 day to about 1 week. In embodiments, the material is exposed to the gas comprising chlorine dioxide for an exposure time of about 1 to 48 hours. In embodiments, the material is exposed to the gas comprising chlorine dioxide for an exposure time of about 1 to 24 hours. In embodiments, the material is exposed to the gas comprising chlorine dioxide for an exposure time of about 1 to 12 hours. In embodiments, the exposure time is about 2 to 12 hours. In embodiments, the exposure time is about 3 to 12 hours.

In embodiments, the RH is at least 5%. In embodiments, the RH is at least 70%. In some embodiments, the fumigation methods disclosed herein are carried out at a relative humidity (RH) in the range of 5% to 80%. In some embodiments, the fumigation methods are carried out at a relative humidity of about 5% to 60%, 5% to 55%, or 5% to 50%, 5% to 45%, or 5% to 40%.

In some embodiments, the solid material comprises a material susceptible to corrosion, e.g., a metal, e.g., iron or an iron alloy (e.g., an iron alloy disclosed herein, e.g., cast iron, carbon steel, or alloy steel).

In some embodiments, the RH is kept low, e.g., below about 70%, 60%, 55%, 50%, 45%, or 40%. In some embodiments, keeping the RH low decreases or prevents corrosion of a solid material that would otherwise occur if the fumigation were performed at a higher RH. In some embodiments, the higher RH is an RH of at least 70%. In some embodiments, the higher RH is an RH above about 70%. In some embodiments, the higher RH is an RH of at least 75%. In some embodiments, the higher RH is an RH above about 75%.

In some embodiments, a method disclosed herein further comprises contacting the solid material with a corrosion inhibitor (e.g., applying a corrosion inhibitor to the surface of the solid material) prior to the fumigating.

In some embodiments, the method further comprises climatizing the enclosed volume in which the fumigating is performed to achieve a desired RH. In some embodiments, the desired RH is an RH in the range of 5% to 80%. In embodiments, the desired RH is an RH of at least 5%. In embodiments, the desired RH is at least 70%. In some embodiments, the method further comprises climatizing the enclosed volume in which the fumigating is performed to achieve an RH below about 70%, 60%, 55%, 50%, 45%, or 40%.

In embodiments, the fumigation methods disclosed herein are carried out at a temperature in the range of about 50° F. to about 175° F. (about 10° C. to 80° C.). In embodiments, the temperature is in the range of about 50° F. to about 100° F. (about 10° C. to about 38° C.). In embodiments, the temperature is in the range of about 60° F. to about 95° F. (about 15° C. to about 35° C. In embodiments, the temperature is at least about 70° F. (at least about 21° C.). In embodiments, the temperature is about 50, 55, 60, 65, 70, 75, 80, 85, 90, 95, 100, 105, 110, 115, 120, 125, 130, 135, 140, 145, 150, 155, 160, 165, 170, or 175° F. (or the corresponding temperature in ° C., which can be calculated using the formula T(° C.)=(T(° F.)−32)/1.8).

In embodiments, the methods comprise climatizing the enclosed volume in which fumigation is carried out, e.g., to achieve a desired RH or RH range (e.g., an RH or RH range disclosed herein) and/or a desired temperature or temperature range (e.g., a temperature or temperature range disclosed herein).

In embodiments, the air flow rate in the enclosed volume is at least about 3 feet per second (ft/sec) (0.9 m/s), for example, at least about 5 feet/sec (1.5 m/s), 10 ft/sec (3 m/s), 15 ft/sec (4.5 m/s), or 20 ft/sec (6 m/s). In embodiments, the air flow rate is 3 to 20 ft/sec (0.9 to 6 m/s). In embodiments, the air flow rate is 5 to 20 ft/sec (1.5 to 6 m/s). In some embodiments, the velocity of the gas stream at or in the vicinity of the material being treated increases due to the circulation of air in the enclosed volume.

In some embodiments, the gas comprises carbon dioxide. In a specific embodiment, the gas consists essentially of carbon dioxide and chlorine dioxide (e.g., chlorine dioxide at a concentration of 1000 to 50,000 ppmᵥ). In a specific embodiment, the gas consists of carbon dioxide and chlorine dioxide.

### Solid Materials

The methods described herein can be used to treat any solid material that is capable of absorbing oil and/or fat. As used herein, a “solid material” can be any solid material that contains an oil and/or fat.

Many solid materials can be exposed to oils and/or fats through normal use, as an incident of normal use, or by accident. In embodiments, the solid material has been exposed to an oil and/or a fat. In some embodiments, the solid material has absorbed the oil and/or the fat. In some embodiments, the solid material has been exposed to an oil and/or a fat and has absorbed the oil and/or the fat.

Some solid materials naturally contain oils and/or fats. For example, certain geologic formations, referred to herein as “hydrocarbon-bearing formations,” naturally contain hydrocarbon compounds, oil, and/or natural gas. In some embodiments, the solid material is a hydrocarbon bearing formation.

In some embodiments, the solid material comprises a crystalline solid. In some embodiments, the solid material comprises an amorphous solid. In some embodiments, the solid material is a crystalline solid. In some embodiments, the solid material is an amorphous solid.

In some embodiments, the solid material comprises a molecular, covalent, ionic, or metallic solid. In some embodiments, the solid material comprises a metallic solid. In some embodiments, the solid material is a molecular, covalent, ionic, or metallic solid. In some embodiments, the solid material is a metallic solid.

In some embodiments, the solid material comprises a metal. In some embodiments, the solid material is a metal.

In some embodiments, the solid material comprises iron. In some such embodiments, the solid material comprises or consists of terra cotta, iron, or an iron alloy. In some embodiments, the iron alloy is cast iron, carbon steel, alloy steel, stainless steel, or high strength low alloy steel.

In some embodiments, the solid material comprises iron or an iron alloy. In some embodiments, the iron or iron alloy is cast iron or steel (e.g., carbon steel, alloy steel, stainless steel, or high strength low alloy steel).

In some embodiments, the iron alloy is cast iron. Cast iron is an iron-carbon alloy with a carbon content greater than 2%. Cast iron can further include silicon (e.g., 1-3% silicon) and/or other components.

In some embodiments, the iron alloy is steel. In some embodiments, the steel is carbon steel, alloy steel, stainless steel, or high strength low alloy steel.

Carbon steel is steel in which the main alloying element is carbon. It typically contains 0.04 to 2% carbon. Steel is considered to be carbon steel when no minimum content is specified or required for chromium, cobalt, columbium [niobium], molybdenum, nickel, titanium, tungsten, vanadium or zirconium, or any other element to be added to obtain a desired alloying effect; when the specified minimum for copper does not exceed 0.40 percent; or when the maximum content specified for any of the following elements does not exceed the percentages noted: manganese 1.65, silicon 0.60, copper 0.60. See www.totalmateria.com/articles/Art62.htm; accessed Dec. 15, 2015. In some embodiments, the carbon steel is a tool steel.

Alloy steel is a steel that contains other alloying elements besides carbon. The other alloying elements are added to improve its properties (e.g., strength, hardness, toughness, wear resistance, corrosion resistance, hardenability, and hot hardness) as compared to carbon steels. Such alloying elements can include, e.g., one or more of manganese, nickel, chromium, molybdenum, vanadium, silicon, boron, aluminum, cobalt, copper, cerium, niobium, titanium, tungsten, tin, zinc, lead, and/or zirconium. In some embodiments, the alloy steel is a tool steel.

Stainless steel is a steel alloy with increased corrosion resistance over that of carbon steel and alloy steel. Typically, stainless steel has a minimum of 10.5% chromium and can include other components, such as, e.g., nickel, carbon, manganese, and molybdenum.

High strength low alloy steel has 0.05-0.25% carbon content and can also include up to 2.0% manganese and small quantities of copper, nickel, niobium, nitrogen, vanadium, chromium, molybdenum, titanium, calcium, rare earth elements, and/or zirconium.

In some embodiments, the solid material comprises rock (e.g., sedimentary rock). In some embodiments, the rock is dolomite, sandstone, limestone, shale, or tar sand. In some embodiments, the solid material comprises dolomite. In some embodiments, the solid material comprises sandstone. In some embodiments, the solid material comprises limestone. In some embodiments, the solid material comprises shale. In some embodiments, the solid material comprises tar sand.

In some embodiments, the solid material comprises sedimentary rock, igneous rock, or metamorphic rock.

In some embodiments, the solid material comprises granite.

In some embodiments, the rock is a hydrocarbon bearing formation. In some embodiments, the hydrocarbon bearing formation comprises dolomite, sandstone, limestone, shale, or tar sand. In some embodiments, the hydrocarbon bearing formation comprises tar sand. In some embodiments, the hydrocarbon bearing formation comprises shale.

In some embodiments, the solid material comprises clay.

In some embodiments, the solid material comprises concrete.

In some embodiments, the solid material comprises brick.

In some embodiments, the solid material comprises wood.

In some embodiments, the solid material comprises plaster.

In some embodiments, the solid material comprises drywall (also known as plasterboard).

In some embodiments, the solid material comprises a ceramic. In some such embodiments, the solid material comprises terra cotta.

In some embodiments, the solid material comprises metal, rock (e.g., sandstone, limestone, shale, or dolomite), clay, concrete, brick, wood, plaster, drywall or a ceramic. In some embodiments, the solid material comprises metal, rock, clay, concrete, brick, wood, plaster, drywall or a ceramic. In some embodiments, the solid material is metal, rock, clay, concrete, brick, wood, plaster, drywall or a ceramic.

### Cleaning Methods

Methods provided herein can be used to clean a solid material. In some embodiments, the solid material is a material used in industry, such as, e.g., a material used in manufacturing, processing, packaging, or transporting of products.

In some embodiments, the solid material is a petroleum tanker. The petroleum tanker can be, e.g., a crude tanker (e.g., an ultra large crude carrier) or a product tanker.

In some embodiments, the methods further comprise removing the drawn out oil and/or fat from the surface of the solid material.

In some embodiments, the removing comprises physically or mechanically removing the oil and/or fat from the solid material. Physically or mechanically removing can be, e.g., by wiping, scraping, or otherwise moving the oil and/or fat off of the surface of the solid material. In some embodiments, physically or mechanically removing the oil and/or fat from the solid material comprises washing the solid material with a washing fluid (e.g., a washing liquid). In some embodiments, the washing fluid comprises or consists of water or an aqueous solution. In some embodiments, the washing fluid comprises or consists of a non-aqueous solvent (e.g., a non-polar organic solvent) or a non-aqueous solution. In some embodiments, the washing fluid comprises a mixture of water and a non-aqueous solvent. In some embodiments, the washing fluid comprises or consists of a flushing medium as disclosed herein.

In some embodiments, the removing comprises applying a chemical to the solid material to remove the oil and/or fat from the solid material. In some embodiments, the chemical is one or more of an alkali (e.g., caustic soda); a surfactant or degreasing agent; and an acid. The chemical can be dissolved in an appropriate solvent (e.g., an aqueous or non-aqueous solvent). An alkali can be used to saponify certain oils and fats (e.g., esters of glycerol and higher fatty acids). The acid can be one or a combination of acids (e.g., organic and/or inorganic acids). Inorganic acids include, e.g., sulphuric acid, nitric acid, sulfamic acid, phosphoric acid, ammonium bifluoric acid, and hydrochloric acid. Organic acids include, e.g., formic acid, citric acid, acetic acid, oxalic acid, EDTA, and DTPA. Chemicals can be applied in steps, optionally with a physical or mechanical removal step (such as, e.g., a washing step) between applications.

The removing can involve other removal methods known in the art.

### Oils and Fats

The methods disclosed herein can draw out an oil and/or a fat from a solid material. In some embodiments, the oil and/or fat is one or more (e.g., a combination) of the oils and/or fats disclosed herein.

The oil and/or fat is typically a substance or combination of substances that is not water soluble or has low solubility in water. In some embodiments, the oil and/or fat has a water solubility of less than or equal to 0.5 g/100 g. In some embodiments, the oil and/or fat has a water solubility of less than or equal to 0.1 g/100 g. In some embodiments, the oil and/or fat includes or is composed primarily of one or more hydrocarbon compounds. In some embodiments, the oil and/or fat is a liquid at 20° C. or has a melting point of 50° C. or less (assuming a pressure of 760 mm Hg). Typically, the oil and/or fat will leave a greasy stain if applied to white paper.

In some embodiments, the oil and/or fat comprises one or more hydrocarbon compounds made up of hydrogen and carbon. In some embodiments, the oil and/or fat consists primarily of hydrocarbon compounds.

In some embodiments, the oil and/or fat comprises a hydrocarbon (e.g., one or more hydrocarbon compounds made up of hydrogen and carbon).

In some embodiments, the oil or fat is a hydrocarbon (e.g., one or more hydrocarbon compounds made up of hydrogen and carbon).

In some embodiments, the hydrocarbon comprises crude oil or natural gas.

In some embodiments, the hydrocarbon is a saturated hydrocarbon, which is also known as an alkane (e.g., a cycloalkane (e.g., a cycloalkane having one ring and the general formula CₙH₂ₙ) or a non-cyclic alkane; a non-cyclic alkane has the general formula CₙH₂ₙ₊₂).

In some embodiments, the hydrocarbon is an unsaturated hydrocarbon. An unsaturated hydrocarbon can be an alkene (e.g., a cyclic alkene or a non-cyclic alkene, e.g., a non-cyclic alkene with one double bond which has the general formula CₙH₂ₙ) or an alkyne (e.g., a cyclic alkyne or a non-cyclic alkyne; a non-cyclic alkyne has the general formula CₙH₂ₙ₋₂).

In some embodiments, the hydrocarbon is an aromatic hydrocarbon, i.e., a hydrocarbon that has at least one aromatic ring. An aromatic hydrocarbon can be, e.g., benzene; toluene; ethylbenzene; xylene (e.g., m-xylene, o-xylene, and/or p-xylene); 1,3,5-trimethylbenzene; or 1,2,4,5-tetramethylbenzene.

In some embodiments, the oil is motor oil (e.g., light motor oil or heavy motor oil).

In embodiments, the oil is a synthetic oil.

In embodiments, the oil and/or fat is a plant-derived oil or fat.

In some embodiments, oil and/or fat is an animal-derived oil or fat.

In embodiments, the oil and/or fat is a cooking oil or fat. A cooking oil or fat can be any plant-derived, animal-derived or synthetic oil or fat used in cooking. Plant-derived oils and fats used in cooking include, e.g., olive oil, palm oil, palm kernel oil, soybean oil, canola oil (rapeseed oil), corn oil, sunflower oil, safflower oil, peanut oil, sesame oil, coconut oil, hemp oil, almond oil, macadamia nut oil, cocoa butter, avocado oil, cottonseed oil, and wheat germ oil Animal-derived oils or fats used in cooking include, e.g., pig fat (lard), poultry fat, beef fat, lamb fat, and fat derived from milk (e.g., butter or ghee).

In some embodiments, the oil and/or fat comprises a fatty acid. In some embodiments, the oil and/or fat comprises a fatty acid ester. In some embodiments, the oil and/or fat is a fatty acid or fatty acid ester.

### Methods of Treating Hydrocarbon Bearing Formations

In a one aspect provided herein is a method of drawing out hydrocarbon from a hydrocarbon bearing formation. The method comprises fumigating the hydrocarbon bearing formation with a gas containing chlorine dioxide, thereby drawing out hydrocarbon from the hydrocarbon bearing formation.

The hydrocarbon bearing formation can include material such as, e.g., dolomite, sandstone, limestone, shale, sand, and/or tar sand.

In some embodiments, the fumigating comprises introducing the gas containing chlorine dioxide into the wellbore of a well that penetrates the hydrocarbon bearing formation. As used herein a “gas containing chlorine dioxide” refers to a predominantly gaseous mixture that includes chlorine dioxide. The mixture can include air and/or other gases in addition to chlorine dioxide.

Typically, the chlorine dioxide is high purity chlorine dioxide and is at least 97, 98, or 99% pure.

In some embodiments, the fumigating is performed as disclosed elsewhere herein.

In some embodiments, the chlorine dioxide is a subcritical gas and is introduced into the wellbore at a concentration such that its partial pressure remains below the explosive limit. Typically, the explosive limit is approximately 83 mmHg. In some embodiments, the chlorine dioxide is introduced at a concentration such that its partial pressure remains below about 50%, 30%, 25%, or 20% of the explosive limit. In some embodiments, the chlorine dioxide is introduced at a concentration such that its partial pressure remains below 42 mmHg, 25 mmHg, 21 mmHg, or 17 mmHg.

In some embodiments, the gas containing chlorine dioxide further comprises carbon dioxide, nitrogen, natural gas, or a combination thereof.

In some embodiments, the gas containing chlorine dioxide comprises carbon dioxide. In some embodiments, the gas containing chlorine dioxide consists essentially of carbon dioxide and chlorine dioxide. In some embodiments, the gas containing chlorine dioxide consists of carbon dioxide and chlorine dioxide. In some embodiments, the carbon dioxide is gaseous carbon dioxide. In other embodiments, the carbon dioxide is supercritical carbon dioxide.

In some embodiments, the gas containing chlorine dioxide comprises nitrogen gas. In some embodiments, the gas containing chlorine dioxide consists essentially of nitrogen gas and chlorine dioxide. In some embodiments, the gas containing chlorine dioxide consists of nitrogen gas and chlorine dioxide.

In some embodiments, the gas containing chlorine dioxide comprises natural gas. In some embodiments, the gas containing chlorine dioxide consists essentially of natural gas and chlorine dioxide. In some embodiments, the gas containing chlorine dioxide consists of natural gas and chlorine dioxide.

In some embodiments, the gas containing chlorine dioxide further comprises hydrogen chloride gas.

In some embodiments, the method further comprises introducing a flushing medium into the hydrocarbon bearing formation (e.g., into the wellbore of the well) and recovering at least a portion of the flushing medium. Typically, the flushing medium is introduced after the fumigating. The flushing medium is a fluid that can be a liquid, a gas, or a mixture thereof. In some embodiments, the flushing medium is mostly liquid and optionally includes some dissolved gas. The flushing medium is suitable for removing drawn out hydrocarbon from the hydrocarbon bearing formation (e.g., a portion of the drawn out hydrocarbon, most of the drawn out hydrocarbon, or substantially all of the drawn out hydrocarbon can be removed). The introduction of the flushing medium is preferably performed before drawn out hydrocarbon can be reabsorbed into the formation. Accordingly, the recovered flushing medium will contain at least a portion of the drawn out hydrocarbon; in some embodiments, the recovered flushing medium will contain most of the drawn out hydrocarbon, or substantially all of the drawn out hydrocarbon. In some embodiments, the flushing is performed immediately after the fumigating.

In some embodiments, the flushing medium comprises produced fluid. In some embodiments, the flushing medium consists essentially of produced fluid. In some embodiments, the flushing medium consists of produced fluid. The produced fluid can be fluid that was previously produced from the well or from another well in the hydrocarbon bearing formation.

In some embodiments, the flushing medium comprises water. In some embodiments, the flushing medium consists essentially of water. In some embodiments, the flushing medium consists of water.

As used herein, the “water” in the flushing medium can be, but is not limited to, fresh water, seawater, produced fluid (which includes mostly water that is produced from a petroleum well along with crude oil and/or natural gas), reclaimed water (e.g., treated or untreated wastewater), or a combination thereof. Accordingly, the water can include other components, such as, e.g., one or more salts, gas, and/or crude oil. In some embodiments, the water is a brine. Wastewater or produced fluid can be reclaimed and treated prior to use in the compositions, methods, and apparatus disclosed herein. Exemplary methods and apparatus for treatment of produced water are described, e.g., in US20140263088 and in WO2014145825. Other known methods of water treatment can also be employed.

As used herein, a “brine” or “brine fluid” is a naturally occurring or artificially created fluid comprising water and an inorganic monovalent salt, an inorganic multivalent salt, or both. An artificially created brine fluid can be prepared using one salt or a combination of two or more salts, as is known in the art. Brines can include chloride, bromide, phosphate and/or formate salts. Examples of salts that can be used in a brine fluid include potassium chloride, sodium chloride, calcium chloride, potassium bromide, sodium bromide, calcium bromide, and zinc bromide. Further examples of salts that can be used in a brine fluid include ammonium chloride, potassium phosphate, sodium formate, potassium formate, cesium formate, ethyl formate, methyl formate, methyl chloro formate, triethyl orthoformate, and trimethyl orthoformate. In some embodiments, the brine includes one or more other added components, such as a viscosifying agent (e.g., a xanthan polymer or hydroxyethylcellulose). In some embodiments, the brine is a “clear brine” that appears clear because it contains few or no suspended solids. In one embodiment, the brine is created by adding salt (e.g., a salt disclosed herein, e.g., KCl) to produced water.

In some embodiments, the flushing medium comprises a non-polar organic solvent. In some embodiments, the flushing medium consists essentially of the non-polar organic solvent. In some embodiments, the flushing medium consists of the non-polar organic solvent.

As used herein, a “non-polar organic solvent” or “organic non-polar solvent” refers to an organic solvent (e.g., a mixture of organic solvents) that has a dielectric constant <5 and that is immiscible (insoluble) in water, or has low solubility in water, as indicated by a water solubility of less than or equal to 0.5 g/100 g. The dielectric constant and solubility in water is typically measured at an ambient temperature of 15 to 30° C., preferably at a temperature of 20° C. Examples of organic non-polar solvents include benzene, cyclohexane, cyclopentane, diesel fuel, ethylbenzene, trimethylbenzene, hexane, heptane, kerosene, pentane, toluene, xylene, and 1,2,4,5-tetramethylbenzene. In some embodiments, the organic non-polar solvent is not soluble in water or has a water solubility of less than or equal to 0.1 g/100 g. Table 1 lists some exemplary organic non-polar solvents.

TABLE 1
Exemplary non-polar organic solvents

| | | Dielectric | | |
|---|---|---|---|---|
| | | constant | | |
| | | (temperature | | |
| | | at which | | Flash |
| | Solubility | measured | | point in |
| Solvent | in Water | in ° C.) | | ° C. |
| pentane | 0.04 g/100 g ⁴ | 1.84 | (20)¹ | −49 ⁶ |
| hexane | 0.01 g/100 g ⁴ | 1.90 | (20)¹ | −26 ⁷ |
| heptane | 0.01 g/100 g ⁴ | 1.92 | (20)¹ | −4 ¹² |
| Benzene | 0.18 g/100 g ⁴ | 2.28 | (20)¹ | −12 ¹³ |
| Cyclohexane | Insoluble ¹¹ | 2.02 | (25) ¹ | −20 ⁸ |
| Cyclopentane | Insoluble ¹¹ | 1.97 | (20)¹ | −37 ¹⁴ |
| Ethylbenzene | Insoluble ¹¹ | 2.44 | (20)¹ | 22 ¹⁵ |
| toluene | Insoluble ¹¹ | 2.39 | (20)¹ | 6 ¹⁶ |
| o-xylene | Insoluble ¹¹ | 2.56 | (20)¹ | 32 ¹⁷ |
| m-xylene | Insoluble ¹¹ | 2.36 | (20)¹ | 27 ¹⁸ |
| p-xylene | Insoluble ¹¹ | 2.27 | (20)¹ | 27 ¹⁰ |
| 1,2,3- | Insoluble ¹¹ | 2.66 | (20) ¹ | 11 ²⁰ |
| trimethylbenzene | | | | |
| 1,2,4- | Insoluble ¹¹ | 2.38 | (20) ¹ | 44 ¹⁹ |
| trimethylbenzene | | | | |
| 1,3,5- | Insoluble ¹¹ | 2.28 | (20) ¹ | 50 ²¹ |
| trimethylbenzene | | | | |
| Kerosene | Generally | 1.8 | (21)² | 38-72° C.⁵ |

| | | | |
|---|---|---|---|
| | Insoluble | | |
| Diesel fuel | Generally | 2.1 ³ | 52 or more⁵ |
| | Insoluble | | |

¹from Table 5.17 of Dean, J. A. (1999) Lange's Handbook of Chemistry, 15^{th} Edition, New York: McGraw-Hill, Inc.
²from www.engineeringtoolbox.com/liquid-dielectric-constants-d_1263.html; accessed Nov. 18, 2015.
³ from www.vega.com/home_tc/-/media/PDF-files/List_of_dielectric_constants_EN.ashx; accessed Nov. 18, 2015. The temperature at which this value was measured was not provided. Because the composition of diesel fuel can vary, the dielectric constant may vary; in any diesel fuel the dielectric constant is expected to be <5.
⁴ www.organicdivision.org/orig/organic_solvents.html; accessed Nov. 18, 2015.
⁵Flash point. (2015, Nov. 7). In Wikipedia, The Free Encyclopedia. Retrieved 23:04, Dec. 4, 2015, from https://en.wikipedia.org/w/index.php?title=Flash_point&oldid=689479169.
⁶ Pentane. (2015, Nov. 16). In Wikipedia, The Free Encyclopedia. Retrieved 23:58, Dec. 4, 2015, from https://en.wikipedia.org/w/index.php?title=Pentane&oldid =690958323.
⁷ Hexane. (2015, Dec. 2). In Wikipedia, The Free Encyclopedia. Retrieved 00:00, Dec. 5, 2015, from https://en.wikipedia.org/w/index.php?title=Hexane&oldid=693378563
⁸ Cyclohexane. (2015, Nov. 20). In Wikipedia, The Free Encyclopedia. Retrieved 00:01, Dec. 5, 2015, from https://en.wikipedia.org/w/index.php?title=Cyclohexane&oldid=691542839.
⁹Ethylbenzene. (2015, Nov. 2). In Wikipedia, The Free Encyclopedia. Retrieved 22:42, Dec. 4, 2015, from https://en.wikipedia.org/w/index.php?title=Ethylbenzene&oldid =688706266
¹⁰ P-Xylene. (2015, Nov. 22). In Wikipedia, The Free Encyclopedia. Retrieved 22:46, Dec. 4, 2015, from https://en.wikipedia.org/w/index.php?title=P-Xylene&oldid=691897047
¹¹ CRC Handbook of Chemistry and Physics, 89^{th} Edition, Edited by David R. Lide, published 2008.
¹² Heptane. (2015, Nov. 22). In Wikipedia, The Free Encyclopedia. Retrieved 00:04, Dec. 5, 2015, from https://en.wikipedia.org/w/index.php?title=Heptane&oldid=691818964
¹³ Benzene. (2015, Dec. 4). In Wikipedia, The Free Encyclopedia. Retrieved 00:05, Dec. 5, 2015, from https://en.wikipedia.org/w/index.php?title=Benzene&oldid=693731378
¹⁴ Cyclopentane. (2015, September 22). In Wikipedia, The Free Encyclopedia. Retrieved 00:07, Dec. 5, 2015, from https://en.wikipedia.org/w/index.php?title=Cyclopentane&oldid=682303646.
¹⁵ Ethylbenzene. (2015, Nov. 2). In Wikipedia, The Free Encyclopedia. Retrieved 00:13, Dec. 5, 2015, from https://en.wikipedia.org/w/index.php?title=Ethylbenzene&oldid=688706266.
¹⁶ Toluene. (2015, Nov. 27). In Wikipedia, The Free Encyclopedia. Retrieved 00:12, Dec. 5, 2015, from https://en.wikipedia.org/w/index.php?title=Toluene&oldid=692661894
¹⁷ O-Xylene. (2015, Nov. 16). In Wikipedia, The Free Encyclopedia. Retrieved 00:17, Dec. 5, 2015, from https://en.wikipedia.org/w/index.php?title=O-Xylene&oldid=690956607.
¹⁸ M-Xylene. (2015, Nov. 16). In Wikipedia, The Free Encyclopedia. Retrieved 00:19, Dec. 5, 2015, from https://en.wikipedia.org/w/index.php?title=M-Xylene&oldid=690955651
¹⁹ 1,2,4-Trimethylbenzene. (2015, Nov. 16). In Wikipedia, The Free Encyclopedia. Retrieved 15:34, Dec. 10, 2015, from https://en.wikipedia.org/w/index.php?title=1,2,4-Trimethylbenzene&oldid=690952112
²⁰ 1,2,3-Trimethylbenzene. (2015, Nov. 2). In Wikipedia, The Free Encyclopedia. Retrieved 15:38, Dec. 10, 2015, from https://en.wikipedia.org/w/index.php?title=1,2,3-Trimethylbenzene&oldid=688696177
²¹ Mesitylene. (2015, July 14). In Wikipedia, The Free Encyclopedia. Retrieved 15:40, Dec. 10, 2015, from https://en.wikipedia.org/w/index.php?title=Mesitylene&oldid=671459559

In some embodiments, the organic non-polar solvent has a flash point of at least 5° C. In some embodiments, the organic non-polar solvent has a flash point of at least 10° C. In some embodiments, the organic non-polar solvent has a flash point of at least 15° C. In some embodiments, the organic non-polar solvent has a flash point of at least 20° C. In some embodiments, the organic non-polar solvent has a flash point of at least 25° C. In some embodiments, the organic non-polar solvent has a flashpoint of at least 30° C.

In some embodiments, the method enhances recovery of hydrocarbon from the well.

In some embodiments, a treatment or method disclosed herein enhances hydrocarbon recovery. A method or treatment disclosed herein is said to “enhance recovery” or to “enhance hydrocarbon recovery” when the application of the method is followed by an increase in the production of total hydrocarbon (crude oil plus natural gas), crude oil, and/or natural gas from a well and/or when the application of the method is followed by an increase in the hydrocarbon cut (e.g., the crude oil cut, the gas cut, or the total hydrocarbon cut of the fluid produced from a well). The “oil cut” refers to the amount of crude oil produced (which can be measured, e.g., in barrels of oil per day (BOPD)) relative to the amount of water produced (which can be measured, e.g., in barrels of water per day (BWPD)) from a well. Similarly, the “gas cut” refers to the amount of natural gas produced relative to the amount of water produced from the well. The “total hydrocarbon cut” refers to the total amount of crude oil and natural gas produced relative to the amount of water produced from a well.

In some embodiments, the increase is an increase of at least about 1, 2, 3, 4, 5, 6, 7, 8, 9, 10, 20, 25, 30, 40, 50, 60, 70, 75, 80, 90 or 100%.

In some embodiments, the increase in hydrocarbon production (e.g., crude oil and/or natural gas production) and/or the increase in hydrocarbon cut (e.g., the oil cut, the gas cut, or the total hydrocarbon cut of the well) is determined based on production values from a period of at least 1 week, 2 weeks, 1 month, 3 months, 6 months, or 12 months following the treatment. The increase can be an increase compared with the corresponding values from a baseline period just prior to the treatment (e.g., a one day, one week, two week, or one month baseline period) and/or from an original drilled production period (e.g., a one day, one week, two week, or one month period following the first production from the well).

In a preferred embodiment, enhanced recovery is indicated by an increase in the average production of hydrocarbon (e.g., crude oil and/or natural gas production) and/or by an increase in the average hydrocarbon cut (e.g., the oil cut, the gas cut, or the total hydrocarbon cut of the well) that is observed based on production values obtained for at least 30 days following treatment compared with production values obtained during a baseline period of 30 days immediately prior to the treatment. In some embodiments, the average production of hydrocarbon (e.g., crude oil and/or natural gas) and/or the average hydrocarbon cut (e.g., the oil cut, the gas cut, or the total hydrocarbon cut of the well) is increased as indicated by production values obtained for at least 1, 2, 3, 4, 5, 6, 7, 8, 9, 10, 11, or 12 months following the treatment compared with production values obtained during a baseline period and/or during an original drilled production period. The well can be a single well that is treated according to a method disclosed herein, or the well can be group of wells in a common formation, wherein one or more of the wells in the group is treated according to a method disclosed herein.

### Decontamination

In some embodiments, a method disclosed herein can decrease, eliminate, and/or inactivate a biological contaminant that was present on or in a solid material prior to exposure of the solid material to chlorine dioxide.

In embodiments, a method disclosed herein decreases or eliminates a biological contaminant that is present on or in the solid material that is treated according to the method, as indicated either by measuring the biological contaminant itself, or an appropriate biological indicator. A biological indicator is an organism other than the biological contaminant that is being targeted by the method that is used as a surrogate for the biological contaminant. The biological indicator is used to assess or verify the efficacy of the method in reducing or eliminating the biological contaminant.

In embodiments, the a method disclosed herein decreases the level of a biological contaminant or biological indicator by at least a 1-log order reduction (“1 log reduction”), a 2-log order reduction (“2 log reduction”), a 3-log order reduction (“3 log reduction”), a 4-log order reduction (“4 log reduction”), a 5-log order reduction (“5 log reduction”), or a 6-log order reduction (“6 log reduction”).

In embodiments, a method disclosed herein is effective to achieve sterilization. As used herein, “sterilizing” or “sterilization” requires at least a 6-log order reduction (“6-log reduction”) of an enumerable biological material (e.g., a biological contaminant or biological indicator).

In some embodiments, a method described herein results in more than a 6-log reduction in the level of a biological contaminant. In embodiments, a method described herein results in at least a 7-log order reduction (“7 log reduction”), an 8-log order reduction (“8 log reduction”), a 9-log order reduction (“9 log reduction”), or a 10-log order reduction (“10 log reduction”) in the level of the biological contaminant.

In some embodiments, a method described herein results in no detectable growth of the biological contaminant. As used herein, a method “eliminates” or results in “elimination” of a biological contaminant when application of the method to a material results in no detectable growth of a biological contaminant that was detected on or in the material prior to application of the method.

All relevant teachings of the documents cited herein are hereby incorporated herein by reference.

### EXAMPLES

### Example 1: Exposing a Core from a Wellbore to Chlorine Dioxide Draws Out Hydrocarbons

To investigate the effect of chlorine dioxide gas on a hydrocarbon bearing formation, a dolomite core taken from a wellbore of an oil and gas well was exposed to chlorine dioxide. The core was cut into approximately 0.5 cm slices. The slices were then broken into halves. Half of each slice was fumigated (experimental slice) and the other half (control slice) was left sitting in the open air as a control. Prior to the fumigation, all of the slices were completely dry and did not release any oil.

For the fumigation, a container was partially filled with an aqueous solution of approximately 4000 ppm (w/w) chlorine dioxide. A rack was placed in the container and the experimental slice was placed on the rack. The experimental slice did not come into contact with the solution. The container was closed so that the liquid chlorine dioxide solution would release chlorine dioxide gas into the headspace. It is estimated that approximately 15,000 ppmᵥ of chlorine dioxide was released into the headspace. The container was kept in the dark, except that the container was taken into the light and opened once per day for 10 days to observe the experimental slice and take pictures. The liquid solution evaporated after 10 days.

The experimental slices showed a uniform visible sheen of oil after 1 day of chlorine dioxide exposure. The experimental slice also turned a reddish color due to oxidation of the iron content of the core. During the course of the 10-day experiment, heavier hydrocarbons began to exude and form localized pools of oil over the sheen. The control slices were completely dry and showed no change over time.

These results show that chlorine dioxide is effective in drawing out hydrocarbon from a hydrocarbon bearing formation. Because it is known that chlorine dioxide in water can be helpful in removing damage from a wellbore, chlorine dioxide dissolved in water has been used in the past to treat damaged wellbores. However, the present result, which shows that an undamaged core exuded hydrocarbons in response to chlorine dioxide gas exposure, was entirely unexpected.

### Example 2: Fumigating Solid Materials with Chlorine Dioxide Draws Out Oils

To investigate the ability of chlorine dioxide to draw out oils from other kinds of solid materials, various solid materials were soaked in various kinds of oils and subsequently exposed to chlorine dioxide. The solid materials that were used were cast iron, stainless steel, and terra cotta. Two samples of each material (an experimental example that was subsequently subjected to fumigation and a control that was subsequently left out in the air) were soaked in light motor oil (SAE 5W20), heavy motor oil (SAE40), heavy mineral oil, lightweight paraffin oil (lamp oil), grapeseed oil, or peanut oil. The terra cotta was soaked overnight (ca. 12 hours). The stainless steel and cast iron were soaked for 1 week.

Prior to the fumigation, the experimental and control samples were wiped off so that no oil could be felt or observed on the surface; the surfaces were dry to touch. For the fumigation, a container was partially filled with 2 gallons of an aqueous solution of approximately 6600 ppm (w/w) chlorine dioxide. A rack was placed in the container and an experimental sample of each material that had been soaked in each type of material (18 experimental samples) was placed on the rack. The experimental samples did not come into contact with the solution. The container was closed so that the liquid chlorine dioxide solution would release chlorine dioxide gas into the headspace. It is estimated that approximately 20,000 ppmᵥ of chlorine dioxide was released into the headspace. The container was kept in the dark for one week without opening the container. The set of 18 control samples were exposed to the ambient air during the one week period.

After the one week fumigation period, the following effects were observed for all types of oils. The surface of the treated cast iron samples had oxidized (rusted) and oil exuded from the material, mixing with the rust to form a paste. The control cast iron samples showed no change and the surfaces felt dry to touch. The treated stainless steel samples exuded oil that formed a continuous layer on the surface. The control stainless steel samples showed no change and the surfaces felt dry to touch. Four of the six experimental terra cotta samples had a consistently visible sheen of oil on the surface. The heavy mineral oil and paraffin lamp oil samples exuded oil in bead-like droplets on the surface. The control terra cotta samples showed no change and the surfaces felt dry to touch. Following the fumigation period, all samples were left out in the laboratory overnight. The next day, the experimental samples had reabsorbed most of the oil.

These results show that chlorine dioxide was effective in drawing out various types of oils from solid materials, including metals and terra cotta.

### Example 3: Fumigating Solid Materials with Chlorine Dioxide Draws Out Fat

To investigate the ability of chlorine dioxide to draw out fat from solid materials, solid materials were soaked in fat and subsequently exposed to chlorine dioxide. The solid materials that were used were stainless steel and terra cotta. Two samples of each material (an experimental example that was subsequently subjected to fumigation and a control that was subsequently left out in the air) were soaked in ghee (clarified butter), which is an animal-derived fat. Two samples of stainless steel and two samples of terra cotta (one sample of each material served as an experimental sample and one sample as a control) were placed in a soaking container filled with ghee and soaked for 24 hours. During the soaking period, the soaking containers were placed in a 105° F. warm water bath to keep the ghee in liquid form. After the soaking period, all of the samples were removed from the container and wiped off so that no ghee could be felt or observed on the surface; the surfaces were dry to touch.

For the fumigation, a container was partially filled with 250 ml aqueous solution of approximately 2500 ppm (w/w) chlorine dioxide. A rack was placed in the container and an experimental sample of each material that had been soaked in the ghee was placed on the rack. The experimental samples did not come into contact with the solution. The container was closed so that the liquid chlorine dioxide solution would release chlorine dioxide gas into the headspace. It is estimated that approximately 7500 ppmᵥ of chlorine dioxide was released into the headspace. The container was kept in the dark for 24 hours without opening the container. The control samples were exposed to the ambient air during the 24 hour period.

After the 24 hour fumigation period, the container was opened and the samples were inspected. Bubbles of ghee appeared on the surface of the fumigated stainless steel and terra cotta samples. The control samples of both materials remained dry and did not exhibit any change in appearance.

These results show that chlorine dioxide was effective in drawing out fat from solid materials, including metal (stainless steel) and terra cotta.

1. A method of drawing out oil and/or fat from a solid material, the method comprising fumigating the solid material with a gas containing chlorine dioxide, thereby drawing out oil and/or fat from the solid material.

2. The method of claim 1, further comprising removing drawn out oil and/or fat from the surface of the solid material.

3. The method of claim 2, wherein the removing is performed during or within 6 hours after the fumigating.

4. The method of claim 2, wherein the fumigating is conducted at a concentration×time (CT) value of 3,000 to 3,000,000 ppmᵥ-hours.

5. The method of claim 2, wherein the solid material has previously been exposed to the oil and/or the fat and has absorbed the oil and/or the fat.

6. The method of claim 5, wherein the method draws out at least 10% by weight of the absorbed oil and/or fat.

7. (canceled)

8. The method of claim 1, wherein the solid material is a metal comprising iron or an iron alloy.

9. The method of claim 8, wherein the iron alloy is cast iron or steel.

10. The method of claim 2, wherein the oil and/or fat comprises hydrocarbon compounds.

11. The method of claim 2, wherein the oil and/or fat is plant-derived or animal derived.

12. (canceled)

13. The method of claim 1, wherein the solid material is a petroleum tanker.

14. The method of claim 13, wherein the method further comprises removing the drawn out oil and/or fat from the surface of the solid material.

15. The method of claim 2, wherein the removing is performed during or within 4 hours after the fumigating.

16. A method of drawing out hydrocarbon from a hydrocarbon bearing formation, the method comprising fumigating the hydrocarbon bearing formation with a gas containing chlorine dioxide, thereby drawing out hydrocarbon from the hydrocarbon bearing formation.

17. The method of claim 16, wherein the fumigating comprises introducing the gas containing chlorine dioxide into the wellbore of a well that penetrates the hydrocarbon bearing formation.

18. The method of claim 16, wherein the gas containing chlorine dioxide further comprises carbon dioxide gas, nitrogen gas, natural gas, or a combination thereof.

19. The method of claim 16, wherein the gas containing chlorine dioxide further comprises hydrogen chloride gas.

20. The method of claim 16, wherein the method further comprises removing the hydrocarbon from the hydrocarbon bearing formation.

21. The method of claim 20, wherein the removing comprises contacting the hydrocarbon bearing formation with a washing fluid.

22. The method of claim 21, wherein the removing comprises introducing a flushing medium into the hydrocarbon bearing formation and recovering at least a portion of the flushing medium.

23. The method of claim 22, wherein the flushing medium is introduced within 4 hours after the fumigating.

24. (canceled)

25. A method of drawing out oil and/or fat from a solid material, the method comprising (i) fumigating the solid material with a gas containing chlorine dioxide, thereby drawing out oil and/or fat from the solid material and (ii) removing drawn out oil and/or fat from the surface of the solid material during or within 4 hours after the fumigating.
